(19) United States
(12) Patent Application Publication
(10) Pub. No.: US 2016/0310048 A1
(43) Pub. Date: Oct. 27, 2016

(54) Implantable Biosensor

(71) Applicant: BIOMICRO PTE. LTD., Singapore (SG)

(72) Inventors: Dexing PANG, Smithtown, NY (US); Qiongzhen LONG, Singapore (SG)

(21) Appl. No.: 15/102,224

(22) PCT Filed: Dec. 5, 2014
(86) PCT No.: PCT/SG2014/000581

§ 371 Date: Jun. 6, 2016

(30) Foreign Application Priority Data
Dec. 5, 2013 (CN) 201320797372.2

(51) Int. Cl. A61B 5/1473; A61B 5/00; A61B 5/07; A61B 5/1486; A61B 5/145

(52) U.S. Cl. CPC A61B 5/1473; A61B 5/14865; A61B 5/14532; A61B 5/14546; A61B 5/07; A61B 5/0031; A61B 2560/0219; A61B 2560/0238

(57) ABSTRACT

The present invention provides a fully implantable device for monitoring at least one physiological parameter of an individual. The device comprises at least one sensor configured to generate a sensor signal representative of the physiological parameter, where each sensor has at least one electrode and at least one membrane adapted to separate the electrode from a medium external to the device. The device also has a programmable chip configured to receive, process and transmit the sensor signal, and a housing adapted to accommodate the sensor and the programmable chip. The present invention further includes a transponder for working with the device and a kit including the device and a means for implantation of the device. Furthermore, the present invention includes a system for monitoring at least one physiological parameter of an individual, the system including a fully implantable device.

### FIELD OF INVENTION

The present invention relates generally to biomedical devices for bio-parametric monitoring, in particular but not limited to fully implantable biomedical devices for bio-parametric monitoring in an individual.

### BACKGROUND OF INVENTION

The following discussion of the background to the invention is intended to facilitate an understanding of the present invention only. It should be appreciated that the discussion is not an acknowledgement or admission that any of the material referred to was published, known or part of the common general knowledge of the person skilled in the art in any jurisdiction as at the priority date of the invention.

According to the statistics of the World Health Organisation (WHO) in 2013, there are currently approximately 360 million diabetics globally. There are about 93 million diabetics in China, 70 million in India, and 23 million in the United States. The number of diabetics increases about 7% annually. The annual medical costs associated with diabetes are about 465 billion US dollars worldwide and 120 billion US dollars in the United States. In USA, the cost of blood glucose test strips alone is as high as 2 billion US dollars every year. Currently, one effective measure to prevent diabetic complications is to timely and accurately monitor the blood glucose level of diabetics.

Blood glucose levels may be monitored by testing blood samples at medical organizations such as hospitals or through the use of a simple blood glucose meter. The main disadvantage of such methods is inconvenience, where blood has to be drawn from the patient each time glucose levels are tested—the drawing of blood is often painful and time-consuming. Further, blood sample testing at hospitals inconveniences patients who are live far from hospitals or patients who become disabled because of diabetic-related complications. Patients often miss the best treatment time because their blood glucose level cannot be monitored timely particularly since glucose levels often fluctuate throughout the day. Finger-pricking methods for drawing of blood is dependent on the patient's skill for accurate testing such that the patient may rely on erroneous data in determining the dosage level of insulin. Moreover, self-monitoring of glucose levels places a significant burden on less capable individuals, such as the young and elderly.

Several products have been developed for the timely monitoring of blood glucose levels. The FreeStyle Navigator® Continuous Glucose Monitor System by Abbott Diabetes Care (approved for marketing in the United States by FDA in 2008) is a small and portable blood glucose monitoring product. The probe of the device is pierced into the human tissue from the outer skin and measures the blood glucose level when the subcutaneous tissue liquid and the blood glucose are in chemical balance. Such a system can only monitor the blood glucose level for a very short period of time (3 days at most), and is very inconvenient for use. The implantable auto-blood glucose-monitor device of DexCom needs an external power supply, has a size about an AA battery, and its implantation is very complicated. The large size of the device increases rate of infection during implantation, which seriously limits its adoption by the market. In addition, the device's lifecycle is also limited by its battery and sensor probe. The GuardianRT system based on CGMS Meditronic measures blood drops, and its other functions and limitations are similar to those of the abovementioned products. The above devices and including many other current implantable blood glucose measuring products on market such as Medtronic MiniMed (CGMS), are not sufficiently small and fully implantable devices. The measuring sensor needs to be replaced every a couple of days, which may cause infections for long-term patients.

Some of existing technologies can only measure the blood glucose level, but not other parameters related with diabetic complications, such as Ketosis and Acid-base imbalance. VERICHIP (covered by U.S. Pat. No. 7,297,112) by Applied Digital Systems (ADS) is an implantable biochemical chip based on RFID technology, and has an integrated temperature sensor module and an RFID tag. However, it does not specifically disclose how multiple biochemical sensors can be integrated to work properly and optimally with the device. The patent does not disclose the biochemical sensor structure, in particular the biochemical sensor structure in the case of multiple sensors, or the technical interactions between the biosensor(s) and RFID baseband chip as to how they should work together. US 2005/0027175 A1 relates to an implantable biosensor, however said publication does not disclose how to include more than one biosensor in an in vivo implantable biosensor or how does the biosensor monitors more than one physiological parameter in vivo. Chinese Patent ZL200720139520.6, owned by Tai Ke Mei Electronic Technology Limited Corp., provides design details of an implantable blood glucose monitoring device based on 130 KHz RFID chip-array and preliminary experiment results. However, the patent does not provide information on how the device can monitor more than one physiological parameter. Further, the core technology disclosed in this patent needs further improvement. US 2013/0211213 A1 discloses an implantable biosensing device which uses LED and photo-luminescent detection techniques. Said device has several disadvantage; firstly, a glowing LED under skin may cause patients to feel uncomfortable having such a device subcutaneously implanted, particularly in social settings; secondly LED consumes a substantial amount of power which can cause inconvenience and further discomfort to patient because frequent charging of the device may be required and the external reader may have to be placed in close proximity to the portion of the body where the device has been implanted in order to wirelessly generate the device's power; and thirdly but most importantly, the device cannot be implanted in a patient's body for a long time because the device is based on a photo-luminescent technology which tends to weaken the sensor's response and the stability after use since the chemical-optical reactions occurring at the sensor, can damage the sensor. Current implantable biochemical parameter monitoring systems which are based on RFID technology, are integrated by micro-processor based RFID chips. In such systems, the biochemical parameter monitoring system is integrated with the core circuits of the RFID chip, hence a system can only monitor a specific parameter. U.S. Pat. No 7,125,382 discloses a blood glucose sensor inside an RFID chip core. The disadvantages of this device is that it can only be equipped with a specific blood glucose monitoring capability and it does not disclose how the device can monitor more than one physiological parameter. It also does not solve biocompatibility issues and high accuracy temperature sensing issues.

Therefore the object of the present invention is to provide for a fully implantable device for bio-parametric monitoring in an individual where the device can monitor more than one physiological parameter.

### SUMMARY OF INVENTION

Throughout the specification, unless the context requires otherwise, the word “comprise” or variations such as “comprises” or “comprising”, are to be construed as inclusive and not exhaustive.

Furthermore, throughout the specification, unless the context requires otherwise, the word “include” or variations such as “includes” or “including”, are to be construed as inclusive and not exhaustive.

In a first embodiment, the present invention provides a fully implantable device for monitoring at least one physiological parameter of an individual, the device comprising at least one sensor configured to generate a sensor signal representative of the physiological parameter, each sensor having at least one electrode and at least one membrane adapted to separate the electrode from a medium external to the device, a programmable chip configured to receive, process and transmit the sensor signal, and a housing adapted to accommodate the sensor and the programmable chip.

Preferably, the membrane is a semipermeable or selectively permeable membrane. It is also preferable that the membrane is impermeable to water molecules.

Preferably, a portion of the membrane in contact with the medium shares a boundary with an external surface of the housing.

Preferably, the housing includes a biocompatible coating, and even more preferably, the coating comprises PEEK or parylene. Other biocompatible coatings commonly used in the art may also be used herein, for example Titanium dioxide (TiO₂).

Preferably, the sensor is an electrochemical sensor. It is preferable that the sensor is a single sensor comprising an auxiliary electrode, a reference electrode and more than one working electrode. It is also preferable that such sensor is configured to detect more than one distinct physiological parameter and generate signals representative and corresponding to each distinct physiological parameter.

Alternatively, the device comprises more than one sensor and each sensor is preferably configured to detect a distinct physiological parameter and generate a sensor signal representative of the distinct physiological parameter. It is preferable that at least one of such sensors is an electrochemical sensor and that such electrochemical sensor may comprise an auxiliary electrode, a reference electrode and more than one working electrode.

Preferably, the sensor includes at least one enzyme. Preferably, at least one of the enzymes is glucose oxidase.

Preferably, the programmable chip is configured to transmit the sensor signal via a wireless communication protocol. It is preferable that the wireless communication protocol is RFID, although it will be understood that other wireless communication protocols may be implemented for the present invention, for example, Wi-Fi and Bluetooth. It is preferred that the RFID is based on a 13.56 mega-hertz RFID standard.

Preferably, the device includes a power supply, where the power in the power supply is preferably wirelessly generated.

Preferably, the device includes a temperature transducer adapted to measure the temperature of the device and generate a temperature measurement signal.

Preferably, the device includes an antenna.

Preferably, the device is implantable in an individual via parenteral and/or enteral means. It would be understood that the device may be implantable in an individual by commonly known methods and means, for example via direct injection to the intended tissue.

In a second embodiment, the present invention provides a system for monitoring at least one physiological parameter of an individual, the system comprising a fully implantable device according to the first embodiment of the present invention, and at least one processor, wherein the device is operable to be in data communication with the processor, the processor is arranged to receive a dataset of physiological parameter of the individual.

Preferably, the processor comprises a reader for receiving a dataset of physiological parameter. Even more preferably, the dataset of physiological parameter is further sent to a central server for further processing and storage.

In a third embodiment, the present invention provides a reader for use with a fully implantable device according to the first embodiment of the present invention, where the reader is in data communication with the fully implantable device.

In a fourth embodiment, the present invention provides a kit comprising a fully implantable device according to the first embodiment of the present invention, and a means for implanting said device in an individual.

### BRIEF DESCRIPTION OF FIGURES/DRAWINGS

The present invention will now be described, by way of example only, with reference to the accompanying drawing, in which:

FIG. 1 provides a schematic representation of the device of the present invention.

FIG. 2 provides a schematic representation of the system of the present invention.

FIG. 3 provides a representation of a one embodiment of the sensor.

FIG. 4 provides a representation of another embodiment of the sensor.

FIG. 5 provides a representation of an antenna of the device of the present invention.

FIG. 6 shows a circuit block diagram of the interface and sensing circuitry as part of the device in accordance with an embodiment of the invention.

Other arrangements of the invention are possible and, consequently, the accompanying drawings are not to be understood as superseding the generality of the preceding description of the invention.

### DETAILED DESCRIPTION OF THE INVENTION

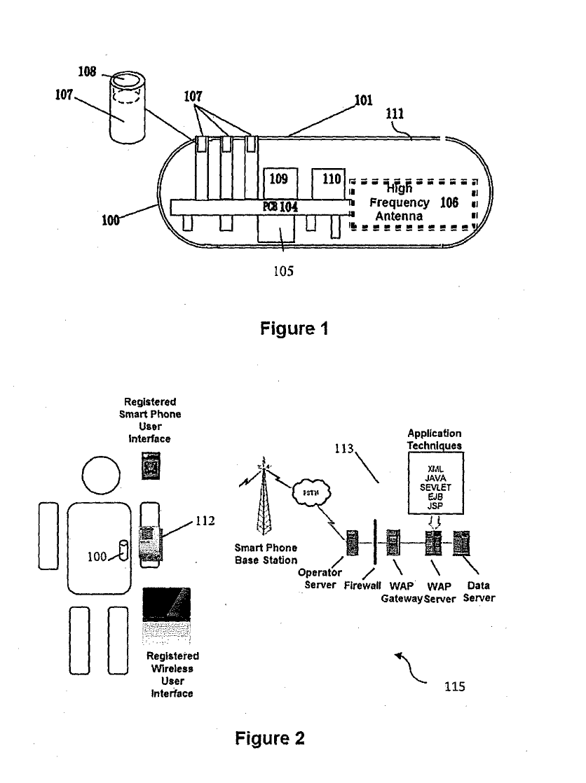

Referring now to the drawings which are for the purposes of illustrating various aspects of the invention and not for purposes of limiting the same, FIG. 1 shows a schematic representation of a fully implantable device of the present invention. The phrase “fully implantable” is taken herein to mean that the device may be placed within the body of an individual without any exposed portions, and can be taken to also mean that the device when fully implanted, is substantially surrounded by cells of the tissue in which it is intended to be placed. The fully implantable device of the present invention is capable of monitoring at least one physiological parameter of an individual. A “physiological parameter” is taken herein to mean a measurable factor of an organism, for example, blood glucose, calcium or cholesterol levels. An “individual' herein refers to an organism and can include but not limited to humans and animals.

In FIG. 1, the fully implantable device 100 includes a housing 111, a biocompatible coating 101, an ASIC module 109, an RFID baseband module 110, PCB 104, power supply 105, high frequency antenna 106, and an electrochemical sensor comprising electrodes 107, membranes 108, and a bioactivator layer (not shown). The electrochemical sensor includes part of PCB 104 which electrically connects the electrodes 107. The device 100 can also include a temperature sensor (not shown) for measuring the temperature of the device and generating a temperature measurement signal. The temperature sensor monitors the temperature of device 100 to calibrate and optimise the signal output which has been found to correlate with temperature. The quality of the signal generated by the device 100 requires a stable reference voltage which is affected by temperature because it is preferable that the integrated circuit of device 100 operates on CMOS band-gap technology which produces a stable voltage closely related to a certain temperature range induced within a narrow P/N band-gap. The stability of the voltage therefore has to be within acceptable fluctuations within the acceptable temperature range within the narrow P/N band gap.

It is understood that power supply 105 does not need to be an active source, such as a battery but can instead derive its power passively.

Membranes 108 reduce the required drive voltage between electrodes 107, in particular the working and reference electrodes, thereby efficiently prolonging the lifetime of the electrochemical sensor. Membranes 108 are also biocompatible and resistant to environmental interferences. Membranes 108 may be fully permeable, semipermeable, selectively permeable or impermeable. Fully permeable membranes allow all molecules to pass through it while semipermeable membranes are membranes which only allow certain molecules to pass through them but not others, where such membranes generally differentiates the molecules based on size. Selectively permeable membranes however selects which types of molecules may pass through, where such selection is typically based on certain factors, for example the charge of the molecule. Impermeable membranes substantially hinder the passing of all molecules through the membrane. In one embodiment, the bioactivator may be located on membranes 108. Alternatively, the bioactivator may be located directly on the surface of electrodes 107 or located on an independent layer in between the membranes 108 and the sensing surface of electrodes 107. In preferred arrangements, the surface of the membranes 108 may be continuous with the housing 111 or the coating 101, i.e. the portion of the membranes 108 which faces the medium external to the device, for example the extracellular matrix or interstitial fluid, may substantially share a same boundary as that of the housing 111 or the coating 101. Alternatively, membranes 108 can, as a single piece or multiple pieces, envelope and cover the external surface of device 100. When the membranes 108 are larger than the opening to the electrodes 107, the layer of membranes 108 may overlay the biocompatible coating 101, if any. It is however understood that the biocompatible coating 101 may also overlay the membranes 108 if membranes 108 are larger than the opening to the electrodes 107. However the biocompatible coating 101 should not in any case, substantially cover the opening to the electrodes 107 so as to obstruct the access of molecules, particularly analytical targets to the electrodes 107. Such arrangements are advantageous because the surface of the device 100 should be sufficiently smooth to faciliate easy implantation of the device. Moreover, in arrangements where the membranes 108 may not be continuous with the housing 111 or coating 101, for example where the membranes 108 are located closer to the electrodes 107 and away from the boundary formed by the external surface of the housing 111 or coating 101, there may be unwanted protein accumulation in the crevices between the housing 111 and membranes 108, which may affect measurement of the physiological parameters since such protein accumulation may cause steric hindrance to incoming analytical targets to the electrodes 107, thereby increasing signal to noise ratio. Membranes 108 have several protective functions—firstly they are biocompatible structures; secondly they provide protection against water, acids and alkalis, and resistance to environmental interferences; and thirdly, by reducing the required drive voltage between electrodes 107, the lifetime of the electrodes can be prolonged, thereby allowing the device 100 to remain implanted for a longer time compared to devices currently known in the art.

In FIG. 1, the electrochemical sensor comprises a portion of PCB 104 (to which the electrode 107 are attached), the electrodes 107, and the membranes 108. The electrochemical sensor measures the desired physiological parameters through electrochemical means, which include but is not limited to amperometric, potentiometric, conductometric and electrical impedance spectroscopy (EIS) sensing methods. Electrochemical means can involve a chemical reaction, typically a catalytic reaction, which results in the production or consumption of electrons that can be measured; or it can involve chemical interactions, such as a binding event between molecules, that causes a shift/change in the electrical dipole or charge of the bound molecules, where such dipole or charge shift/change can be measured. Such measurands can be generally referred to as signals. The electrochemical sensor may be a two (2) electrodes or three (3) electrodes system as commonly understood in the art. There are two electrodes in a two (2) electrodes system, a working electrode and an auxiliary electrode, both electrodes form a circuit where a current through the circuit is detected/measured. The auxiliary electrode can act as a cathode when the working electrode operates like an anode, and vice versa. In a three (3) electrode system, there is an additional reference electrode which provides a reference potential for the working electrode. The electrodes 107 are made from material commonly known in the art, for example, the working and auxiliary electrodes may be made from carbon while reference electrodes can be made from Ag/AgCl₂. It is advantageous to use electrochemical sensors for device 100 because such sensors have innate sensitivity and simplicity. Further, the signals generated can be easily amplified through the use of suitable electronic components, for better information and data on the patients. Furthermore, electrochemical sensors can be easily incorporated in device 100 and they work well together with membranes 108 to accurately and sensitively measure the desired physiological parameters. However, a skilled person may contemplate the use of other types of sensors in place of electrochemical sensors, such as optical, acoustic and colorimetric sensors. However, such sensors are not useful in the current implementation because such sensors are usually not as sensitive compared to electrochemical sensors, they will likely complicate the internal architecture of the implantable device when installed and the data obtained may not be reliable, for example the acoustic wave in acoustic sensors may be absorbed by surrounding structures, thereby affecting the reliability of the signal.

The electrochemical sensor in FIG. 1 is preferably an amperometric sensor where targets undergo a catalytic chemical reaction (usually at the working electrode) to produce electrons which creates a current in the sensor's circuit, whereby the generated current intensity is proportional to the concentration of the measured targets. The current is measured and processed by ASIC module 109 which communicates with the RFID baseband chip 110. The RFID baseband chip 110 receives from the ASIC module 109 data/information on the current measured and transmits such data via the high-frequency antenna 106 to a reader 112. In this case, the signal which is the current being measured, is an analog signal—however depending on the application, a skilled person would know that the signal can also be a digital signal.

A bioactivator is understood to refer to an entity which can cause or alter the speed of a chemical reaction where the products of the reaction can be used in the detection and measurement of the desired physiological parameter. A bioactivator can include but is not limited to biological and non-biological catalysts. An example of a non-biological catalyst is a photocatalyst which catalyses a reaction upon light exposure—however devices utilising photocatalysts have short lifespans and the design structure of such devices is usually complicated. A biological catalyst is preferred and an example of a biological catalyst is an enzyme which catalyses a reaction to produce electrons to generate a current in the electrodes 107, the current being representative of the physiological parameter being measured. Different bioactivators can be used in the measurement of different targets, for example glucose oxidase for blood glucose, ATPase for aerobic metabolism, lactate oxidase for calcium abundance in the bones, and lipoxygenase for cholesterol level. ASIC module 109 maintains the synchronous or asynchronous measuring functionalities for physiological parameters. Measurement of different physiological parameters can be done by simply changing all electrodes 107, changing the working electrodes only, or changing the bioactivators at the electrodes 107, and the measuring accuracy can be improved by repeated measurements. Blood glucose may be first measured as the most important physiological parameter for diabetes and thereafter blood lipids—accordingly, the main measuring target is set as blood glucose, and accordingly the main bioactivator is glucose oxidase. ASIC module 109 is configurable so as to adapt to the measuring of different physiological parameters, since it would be understood that there are different acceptable ranges associated/correlated to different physiological parameters, for example in humans, the normal preprandial blood glucose level according to the American Diabetes Association is 70-130 mg/dL (approximately 3.9-7.2 mmol/L); the desirable total blood cholesterol level according to the National Heart Centre Singapore is less than 200 mg/dL (approximately 5.5 mmol/dL); and the normal human body temperature range is 36.5-37.5° C.

The biocompatible coating 101 resists moisture, acids and alkalis and provides biocompatibility such that the device 100 does not elicit a toxic or immunogenic effect, or damages surrounding tissues when implanted. Accordingly, a device or material is considered biocompatible when said device or material is able to elicit an appropriate biological response in a specific application without producing a toxic, injurious or immunogenic response in living tissue, where eliciting an appropriate biological response can include not eliciting a biological response at all. Preferably parylene or PEEK (polyether ether ketone) material is used as the biocompatible coating. Parylene or PEEK not only provides good mechanical and chemical performance, such as tensility resistance, pressure resistance and corrosion resistance, but are also one of the best harmless medical materials currently available. Biocompatible coating 101 is applied everywhere on the device except where membranes 108 are located.

The device 100 may be implanted via parenteral and/or enteral means, which include but are not limited to intramuscular, intravenous, subcutaneous, oral or transdermal means. Preferably, the device 100 is implanted via direct injection into the target tissue. Implantation of the device 100 may be achieved by means commonly known in the art, for example if by way of injection, the device 100 may be implanted under an individual's epidermal layer at a depth of 2-3 mm from the surface of the skin using a standard medical syringe and a stainless steel needle with an inner diameter of 2 mm. The device 100 may also be implanted using a specially fabricated implantation device, where the device 100 and the implantation device may together form a kit.

Turning to FIG. 2, said figure shows the fully implantable device 100 having been implanted in an individual, the device 100 being part of a system 115, where the device 100, based on RFID/ASIC technologies, is combined with passive high-frequency sensing technology and networking technology. Data relating to the physiological parameters which has been collected by the device 100, is wirelessly transmitted to a reader 112 which processes and transfers said data to an online server 113. It is understood that the reader 112 can transmit the data via wired or wireless means to the online server 113. The data is accessible by patients and medical personnel so as to enable efficient and timely monitoring of a patient's physiological parameters and treatment of the patient at a time when the physiological parameter of the patient being monitored does not meet the normal acceptable physiological range. For example, the normal preprandial blood glucose level for humans according to the American Diabetes Association is 70-130 mg/dL—therefore if a patient's preprandial blood glucose level exceeds this range, the relevant medical personnel may be alerted. A 13.56 MHz high frequency RFID standard is preferably adopted for the device, which reduces the physical size of the antenna 106, makes the device 100 more compact with higher gain, simplifies the production process and reduces the cost of manufacture of the device 100. The device 100 may actively measure the physiological parameters and transmit the data related to such measurements to the reader 112 when said reader is brought close to the device 100. Alternatively and preferably, the reader 112 having an RFID transceiver can initialise a communication process with device 100 by generating a signal to instruct device 100 to begin measuring the desired physiological parameter for a corresponding signal to be generated and transmitted. When activated by the reader 112 through a power conversion step and through a signal request for data collection, the device 100 begins to activate the sensor's analog front end and the interface circuitry to collect data on the desired physiological parameters and thereafter transmit such data to the reader 112.

Device 100 can also work, wired or wirelessly, with secondary device or system implanted in a patient's body, where the secondary device or system may exert a homeostatic effect on the patient when the device 100 detects that the monitored physiological parameter of the patient fails to meet the normal acceptable physiological range. Exerting a homeostatic effect includes but is not limited to inducing a change to a patient's abnormal physiological parameter, for example through the use of drugs, to a normal stable state. In the case of diabetic patients, device 100 may be in wired or wireless communication with an artificial pancreas system implanted in a patient's body. This is in contrast to traditional applications where artificial pancreas systems obtain information regarding a patient's blood glucose level from tests done outside of the patient's body which usually involve drawing the patient's blood. Advantageously with the present invention, device 100 can alert the artificial pancreas system to release an appropriate amount of insulin to correct abnormal blood glucose levels of the patient when said levels go beyond the normal physiological range.

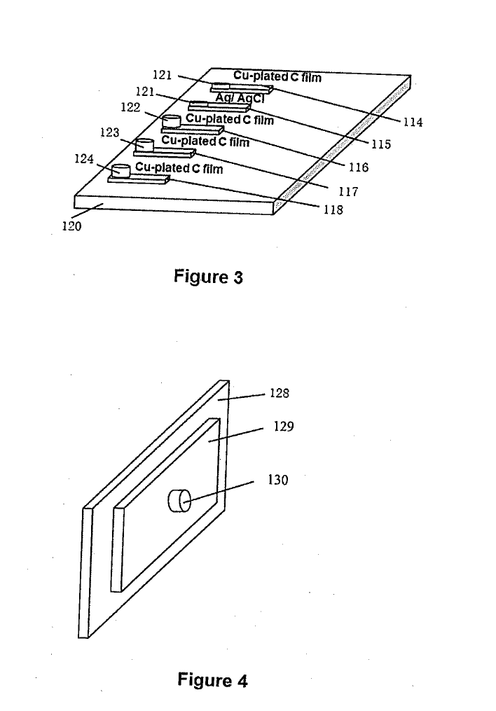

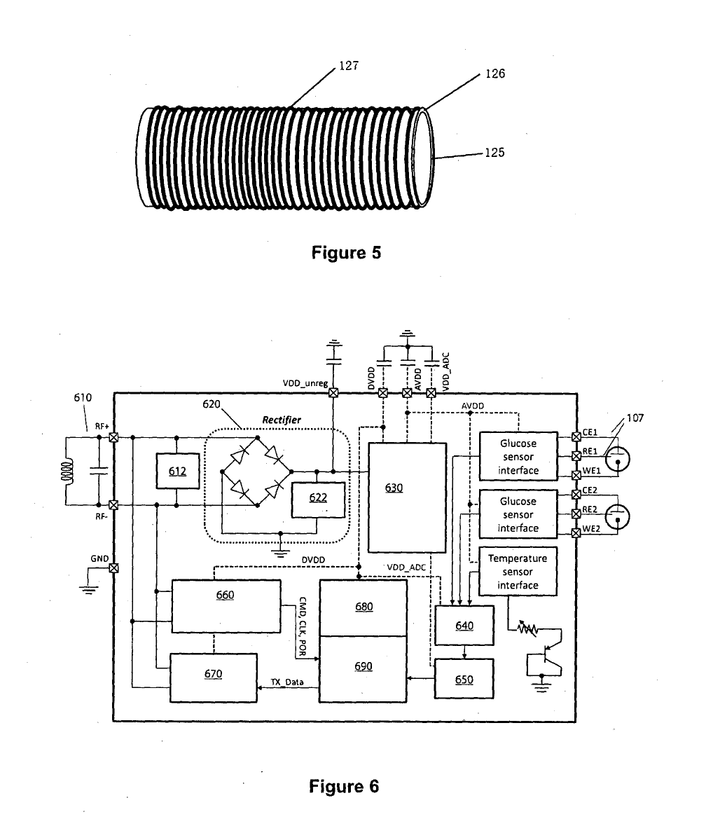

Different embodiments of the electrochemical sensor will be described in more detail in FIGS. 3 and 4. In one embodiment of the electrochemical sensor as shown in FIG. 3, the sensor includes a PCB base 120, an auxiliary electrode 114, a reference electrode 115, multiple working electrodes 116, 117 for the same type of targets (e.g. blood glucose), and a working electrode 118 for a target different from that detected by working electrodes 116, 117 (e.g. lipids). Semipermeable membranes 121 to 124 are provided on the electrodes, where membranes 122 and 123 include bioactivators for detecting same target, for example the bioactivator located on membranes 122 and 123 is glucose oxidase, while the membrane 124 includes a bioactivator for detecting another target, for example the bioactivator located on membrane 124 is lipoxygenase. By having two electrodes 116, 117 detecting the same target, the number of measurements relating to the same physiological parameter increases, thereby resulting in a more reliable output signal with better accuracy. Alternatively, these electrodes 116, 117 may work in an alternating manner, whereby only one electrode 116 or 117 is conducting the measurement of the intended physiological parameter at one time, thereby prolonging the life span of the electrodes 116, 117. The electrodes 107 in FIG. 1 are analogous to the auxiliary electrode 114, reference electrode 115, and working electrodes 116, 117, 118. The membrane 108 is analogous to membranes 122, 123 and 124. In this arrangement, the ASIC module 109 utilises the auxiliary electrode 114, the reference electrode 115, working electrodes 116 and 117 which have the same bioactivators on membranes 122 and 123 respectively and the working electrode 118 which has the bioactivator on membrane 124 to measure heterogeneous targets simultaneously or in series. The ASIC module 109 can also utilise the auxiliary electrode 114, the reference electrode 115, and the working electrodes 116 and 117 having the same bioactivators on membranes 122 and 123 respectively, to measure homogeneous targets simultaneously or in-series. For example, glucose oxidase can be provided on membranes 122 and 123 on multiple working electrodes 116 and 117 respectively, which are in turn used to collect blood glucose information. On the other hand, the heterogeneous measuring target is set as blood lipid, and data on blood lipid levels is collected through the working electrodes 118 which has the bioactivator on membrane 124, for detecting heterogeneous targets.

In another embodiment of the electrochemical sensor for device 100, FIG. 4 shows a working electrode 128 with a bioactivator layer 129 covered on the working electrode 128. The bioactivator layer 129 comprises osmium metal complex and glucose oxidase, which can reduce the drive voltage between the working electrode and the reference electrode and prolong the lifetime of the biochemical sensing materials. Reduction-oxidation (redox) reactions occur at electrode 128 and such reactions would be well understood by a person skilled in the art. However for the avoidance of doubt, the chemical reactions of this process taking place at the working electrode 128 for this embodiment are represented as follows:

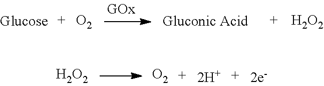

Accordingly, the amount of electrons transferred during the redox process to electrode 128, is proportional to glucose concentration.

A selectively permeable membrane 130 is provided on top of the bioactivator layer 129. The selectively permeable membrane 130 is impermeable to water, i.e. it can stop the penetration of water molecules, and it permits the diffusion of glucose to the electrode 128 at a reduced rate by a factor of approximately 50. Hence, during the redox process, the number of electrons transferred is effectively suppressed in the sensing circuit, and the working voltage is greatly reduced. Due to the existence of the bioactivator layer 129, the redox process does not rely on the extra oxygen molecules outside of the redox system. Such a characteristic ensures stable output of blood glucose data from the working electrode. In this, embodiment, the working area of the sensor is around 0.15 mm², and the measuring accuracy of 0.1 nA/(mg/dL) can be achieved for the blood glucose density of 20-500 mg/dl. Preferably, the potential between working and reference electrodes is 40 mV.

FIG. 4 shows the architecture of the coils of the antenna 106, which is a high-frequency antenna. Antenna 106 may be produced using chemical or physical manufacturing processes as known to a skilled person. In this embodiment, the copper coils 127 of antenna 106 are formed by applying a high dielectric constant oxide ceramics 126 (e.g. Al₂O₃) on high frequency ferrite cores 125. Such architecture can improve the system stability and applicability, and reduce the production cost as well. The physical size of the antenna 106 is reduced because the ferrite with high permeability is adopted for the core of the antenna 106. The coils 127 can provide power supply for the system by inducing the high frequency electromagnetic fields triggered by the reader 112, and serve as the data transceivers for the device 100 as well. When the reader 112 and device 100 are placed close to each other, the antenna 106 of the device generates the power by electromagnetic induction to start-up the device 100. Thereafter, the acquired information from the device 100 is transmitted to the reader 112 wirelessly. As the system adopts high-frequency RFID design principles, the antenna 106 of the device 100 can be processed through chemical etching. With this approach, the stability of the system 115 can be improved while the manufacturing cost can be reduced.

FIG. 6 shows an example of an interface circuit and sensing circuit of the device 100 implemented in the form of an electronic chip, which may be or form part of the PCB 104. The interface circuit and sensing circuit comprises a RF antenna 610 operable to receive/send RF input/output (in the form of data packets) from/to reader 112; a rectifier 620 operable to rectify the received RF input; a power management module 630 operable to receive rectified RF input, a portion of the RF input used for powering the module 630. Upon power up, the power management module 630 is further operable to:
a. provide drive voltages AVDD for driving the electrodes 107;
b. provide drive voltage VDD_ADC for driving an analogue to digital convertor 650; and
c. provide drive voltages DVDD for driving other components such as signal demodulator, clock 660; multiplexer 640; load modulator 670; EEPROM 680 and digital baseband module 690 etc.

RF limiter 612 may be placed in parallel with the RF antenna 610 for RF circuit protection. Likewise the rectifier may comprise voltage limiter 622 for circuit protection.

In operation, the measurement signals obtained from the glucose sensor interfaces and temperature sensor interface are multiplexed and converted to a digital data packet for feeding into the digital baseband module 690, which converts the digital data packet into a transmission data packet to be sent to the reader 112. The transmission data packet may be sent to a load modulator for signal modulator before being sent to the reader 112 via the RF antenna 610.

In accordance with another embodiment of the invention the reader 112 may be embedded in a mobile device, such as a mobile smartphone device. The mobile device may comprise a dedicated software application installed thereon for the purpose of enabling data communication between the reader 112 and the device 100. Mobile device may further comprise the necessary user interface for activating the device 100 to collect measurements of the physiological parameter of the individual and thereafter to collect the data transmitted from the device 100.

It is to be understood that the above embodiments have been provided only by way of exemplification of this invention, such as those detailed below, and that further modifications and improvements thereto, as would be apparent to persons skilled in the relevant art, are deemed to fall within the broad scope and ambit of the present invention described. In particular, the following additions and/or modifications can be made without departing from the scope of the invention:
The electrochemical sensors may be electronically arranged in series or parallel.
The materials forming the electrodes can include carbon, graphene, glassy carbon, and noble metals such as gold and platinum.
The electrodes may be arranged in any particular manner in the device so long as the electrodes can have access to their intended targets for measuring the relevant physiological parameter.
The thickness, hydrophilicity, hydrophobicity, charge of the membrane may vary depending on the applications of the device of the present invention.

Furthermore, although individual embodiments have been discussed it is to be understood that the invention covers combinations of the embodiments that have been discussed as well.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

1. A fully implantable device for monitoring at least one physiological parameter of an individual, the device comprising:
a. at least one sensor configured to generate a sensor signal representative of the physiological parameter, each sensor having at least one electrode and at least one membrane adapted to separate the electrode from a medium external to the device;
b. a temperature transducer adapted to measure the temperature of the device and generate a temperature measurement signal;
c. a programmable chip configured to receive, process and transmit the sensor signal and temperature measurement signal; and
d. a housing adapted to accommodate the sensor and the programmable chip,
wherein the sensor signal is capable of being calibrated and optimized against the temperature measurement signal.

2. The fully implantable device of claim 1, wherein the membrane is a semipermeable or selectively permeable membrane, wherein the membrane is impermeable to water molecules and wherein a portion of the membrane in contact with the medium substantially shares a boundary with an external surface of the housing.

3. (canceled)

4. (canceled)

5. The fully implantable device of claim 1, wherein the housing includes a biocompatible coating and wherein the biocompatible coating comprises PEEK or Parylene.

6. (canceled)

7. The fully implantable device of claim 1, wherein the sensor is a single electrochemical sensor comprising an auxiliary electrode, a reference electrode and more than one working electrode, and wherein the sensor is configured to detect more than one distinct physiological parameter and generate sensor signals representative and corresponding to each distinct physiological parameter.

8. (canceled)

9. (canceled)

10. The fully implantable device of claim 1, the device comprising more than one sensor, wherein each sensor is configured to detect a distinct physiological parameter and generate a sensor signal representative of the distinct physiological parameter and wherein at least one sensor is an electrochemical sensor.

11. (canceled)

12. (canceled)

13. The fully implantable device of claim 1, the sensor including at least one enzyme, wherein the enzyme is glucose oxidase.

14. (canceled)

15. The fully implantable device of claim 1, the programmable chip configured to transmit the sensor signal via a wireless communication protocol, wherein the wireless communication protocol is RFID and wherein the RFID is based on a 13.56 mega-hertz RFID standard.

16. (canceled)

17. (canceled)

18. The fully implantable device of claim 1, the device including an antenna and a power supply, wherein power in the power supply may be wirelessly generated.

19. (canceled)

20. (canceled)

21. (canceled)

22. A processor for use with a fully implantable device according to claim 1, wherein the processor is operable to be in data communication with the fully implantable device.

23. The processor of claim 22, wherein the sensor signal is capable of being calibrated and optimized against the temperature measurement signal by the processor.

24. (canceled)

25. A system for monitoring at least one physiological parameter of an individual, the system comprising:
a fully implantable device comprising:
a. at least one sensor configured to generate a sensor signal representative of the physiological parameter, each sensor having at least one electrode and at least one membrane adapted to separate the electrode from a medium external to the device;
b. a temperature transducer adapted to measure the temperature of the device and generate a temperature measurement signal;
c. a programmable chip configured to receive, process and transmit the sensor signal and temperature measurement signal;
d. a housing adapted to accommodate the sensor and the programmable chip; and
at least one processor,
wherein the sensor signal is capable of being calibrated and optimized against the temperature measurement signal and wherein the device is operable to be in data communication with the processor, and the processor is arranged to receive a dataset of physiological parameter of the individual.

26. The system of claim 25, wherein the processor is configured to receive, calibrate and optimize the sensor signal against the temperature measurement signal.

27. The system of claim 25, wherein the processor comprises a reader for receiving a dataset of the physiological parameter of the individual, and wherein the dataset of physiological parameter is further sent to a central server for further processing and storage.

28. (canceled)

29. The system of claim 25, wherein the membrane is a semipermeable or selectively permeable membrane, wherein the membrane is impermeable to water molecules, and wherein a portion of the membrane in contact with the medium substantially shares a boundary with an external surface of the housing.

30. (canceled)

31. (canceled)

32. The system of claim 25, wherein the housing includes a biocompatible coating, and wherein the biocompatible coating comprises PEEK or Parylene.

33. The system of claim 25, wherein the sensor is a single electrochemical sensor comprising an auxiliary electrode, a reference electrode and more than one working electrode, and wherein the sensor is configured to detect more than one distinct physiological parameter and generate sensor signals representative and corresponding to each distinct physiological parameter.

34. (canceled)

35. (canceled)

36. The system of claim 25, the device comprising more than one sensor, wherein each sensor is configured to detect a distinct physiological parameter and generate a sensor signal representative of the distinct physiological parameter, and wherein at least one sensor is an electrochemical sensor.

37. (canceled)

38. (canceled)

39. The system of claim 25, the sensor including at least one enzyme, and wherein the enzyme is glucose oxidase.

40. The system of claim 25, the programmable chip configured to transmit the sensor signal via a wireless communication protocol, wherein the wireless communication protocol is RFID, and wherein the RFID is based on a 13.56 mega-hertz RFID standard.

41. (canceled)

42. The system of claim 25, the device including an antenna and a power supply, and wherein power in the power supply may be wirelessly generated.

43. (canceled)

44. (canceled)
